(19) United States
(12) Patent Application Publication
(10) Pub. No.: US 2009/0175924 A1
(43) Pub. Date: Jul. 9, 2009

(54) COATED STENT

(75) Inventor: Matti Siren, Helsingfors (FI)

Correspondence Address: MORRISON & FOERSTER LLP, 425 MARKET STREET, SAN FRANCISCO, CA, 94105-2482, US

(73) Assignee: BIONERIS AB, Lidingo (SE)

(21) Appl. No.: 12/405,192

(22) Filed: Mar. 16, 2009

Related U.S. Application Data
(62) Division of application No. 10/526,690, filed on Mar. 3, 2005.

(30) Foreign Application Priority Data
Sep. 3, 2002 (FI) 20021570

(51) Int. Cl. A61F 2/00; A61K 31/66

(52) U.S. Cl. 424/423; 514/75

(57) ABSTRACT

A method of implanting a stent whereby the risk of restenosis is reduced, the method including the step of implanting into a patient susceptible to post-implant restenosis a stent coated with a therapeutically effective amount of a compound for reducing the onset or severity of restenosis, the compound containing a high density, negatively charged domain of at least three vicinally oriented phosphorus-containing radicals.

### CROSS-REFERENCE TO RELATED APPLICATIONS

This application is a Divisional of U.S. patent application Ser. No. 10/526,690, filed Mar. 3, 2005, which is a U.S. National Stage Application of International Application No. PCT/SE2003/001360, filed Sep. 2, 2003, which claims priority to Finnish Application No. FI 20021570, filed Sep. 3, 2002, which are hereby incorporated by reference in their entirety.

The present invention relates to an improved construction of coated surgical stent with a compound containing a high density, negatively charged domain of at least three vicinally oriented phosphorus-containing radicals, such device being defined as an object used for example to hold open blood vessels dilated by angioplasty, particularly coronary blood vessels, iliac aretrias and the alike and which device are utilized to widen blood vessels or other orifices in the body and which compound is preventing restenosis.

Procedures involving the use of balloon dilatation catheters, stents and other percutaneously delivered interventional devices are commonly performed on patients with coronary heart disease (CHD). The balloon angioplasty is the strategy of choice in the majority of cases and the procedure is often followed by stenting. Despite the fact that centres undertaking such procedures are properly equipped and staffed, restenosis, i.e. reocclusion of the vessels, is occurring in 30-50% of the patients treated.

In 1995 nearly 300,000 percutaneous coronary interventions were carried out in Europe and the corresponding figure for the US was approximately 500,000 and these figures continue to increase world wide. Another 500,000 coronary artery bypass procedures were performed in the US. The average cost is more than 20,000 USD per procedure. The costs for the health care system to repeat angioplasty procedures to treat restenosis is estimated to exceed USD 2.5 billion annually.

Restenosis is considered to be one of the major limitations of percutanous transluminal coronary angioplasty. Risk factors for the development of restenosis are multifactoral and mostly unknown. The traditional atherosclerotic risk factors such as hypertension, smoking and cholesterol have not been associated with restenosis. The mechanism for the process of restenosis involves many factors including vasoconstriction, migration and proliferation of smooth muscle cells, the release of regulatory substances such as growth factors, synthesis of extracellular matrices, neointimal formation and remodelling of vessels.

In surgical and other related invasive medicinal procedures, the insertion and expansion of stent devices in blood vessels, urinary tracts or other difficult to access places for the purpose of preventing restentosis, providing vessel or lumen wall support or reinforcement and for other therapeutic or restorative functions have become a common form of long-term treatment. These devices are designed primarily as permanent implants which may become incorporated in the vascular or other tissue which they contact at implantation.

Stents are generally cylindrical and perforated with passages that are slots, ovoid, circular or the like shape. Stents may also be composed of helically wound or serpentine wire structures in which the spaces between the wires form the passages. Furthermore, stents may be flat perforated structures that are subsequently rolled to form tubular structures or cylindrical structures that are woven, wrapped, drilled, etched or cut to form passages. Examples of stents are described for example in U.S. Pat. Nos. 4,733,665, 4,800,882, 4,886,062 and 5,514,154.

These stents can be made of biocompatible materials including biostable and bioabsorbable materials. Suitable biocompatible materials include for example stainless steel, tantalum, titanium alloys and cobalt alloys. Suitable non-metallic biocompatible materials include polyamides, polyolefins and polyesters.

Implanted stents have also been used to carry medicinal agents, such as thrombolytic agent. For example U.S. Pat. No. 5,092,877 discloses a stent of a polymeric material which may be employed with a coating associated with the delivery of drugs. Coatings are often based on film-forming polymers which can be absorbable or non-absorbable and must be biocompatible in order to minimize irritation of the vessel wall. The polymer may be either biostable or bioabsorbable but mostly a bioabsorbable polymer is preferred since it will not be present long after impantation to cause any adverse, chronical response. Suitable film-forming bioabsorbable polymers that could be used are for example aliphatic polyesters, poly(amino acids), copoly(ether-esters), polyalkylenes oxalates, polyamides, poly(iminocarbonates), polyorthoesters, polyoxaesters, polyamidoesters, polyoxaesters, poly(anhydrides), polyphosphazenes, biomolecules and blends thereof.

The polymers used for coatings must be film-forming polymers that have molecular weight high enough not to be waxy or tacky. Furthermore, the polymers must adhere to the stent and not be so deformable that they will be displaced by hemodynamic stresses. The polymers should be chosen in such a way that they will not be rubbed off during handling or deployment of the stent and must not crack during expansion of the stent.

Other delivery methods of active agents have also been described. For example, the active compound can be entrapped into the metal of the stent which has been modified to contain micropores or channels. Methods such as laser ablation techniques could also be utilized. Furthermore the active agent could be covalently bonded to the stent via different chemical techniques such as the formation of esters, amides and anhydrides. The stent could also be inserted into a sleeve or mesh which is comprised or coated with the active agent.

Typically, the stent is delivered to the desired location in the body via an inflatable balloon to which the stent is attached. When the balloon is inflated, the stent expands thereby widening the orifice. Other mechanical devices which cause the expansion of the stent are also utilized. Such devices usually comprise a tubular cage structure which is inserted in a collapsed state into a blood vessel, or other tubular structure and thereafter expanded using a balloon catheter or using for example the self expanding properties of the alloy which the stent is constructed of.

According to the invention claimed in PCT/SE 02/01016, there is described the use of a compound containing a high density, negatively charged domain of vicinally oriented radicals for the preparing of a medicament for preventing, alleviating or combatting restenosis in mammals including man. With the above state of the art in mind it has been realized that the usage taught in PCT/SE 02/01016 can be utilized in an especially effective manner by incorporating the descirbed compounds as coatings on surgical stents by methods known per se.

One objective of this invention is to provide a stent which is capable of delivering a therapeutic agent over a period of time. Such an agent might be directed specifically at the preventing of the proliferation of smooth muscle cells locally at the place of the stent in the lumen wall. The agent or agents may be retained in the coating, slits or interstices of the device to be diffused over a period of time following the insertion of the stent into the blood vessel or other tubular structure.

The ideal function of a stent can be described as follows. The objective of applying stents is to ensure that while the narrowing of the lumen is removed as completely as possible, the lumen does not experience a renarrowing of the vessel wall, a phenomenon known as restenosis. The tissue damage caused by the dilation of the vessel wall results in excessive cell proliferation. The resulting phenomenon can be described in terms of hyperplasia and hypertrofia. There have been many attempts to prevent these negative processes but even in the best scenarios only 60-70% of the cases have given satisfactory results in terms of re-narrowing after six months. Furthermore it is to be noted that when restenosis of blood vessles has been prevented by other compounds such as cytostatic substances a plethora of side effects has been observed.

The optimal situation can be described by setting the following targets for the treatment:
The treatment should be applied in a focused manner on a limited area.
The intensity and efficacy of the treatment should be controlled.
The time period of the treatment should be between 7 and 30 days.
The effect of the treatment should apply only to the damaged tissue and paracrinic cell population.
The effect of the treatment should not advance or hinder the coagulaton cascade.
The active substance should not influence the intact cells in the periferic blood circulation.
The active substance should be metabolized into atoxic by-products.
The active substance should retain its therapeutic properties while attached to the stent.
The active substance should prevent the formation of neointima.
The active substance should prevent the formation of restenotic lesions on the border of between the stent and the untreated portion of a vessel or tubular wall.
The active substance should not be absorbed through intact cell walls.
The active substance should not be antigenetic.

According to the present invention it has surprisingly become possible to prepare a coated stent for implantation in human vessels, orifices and conduits for creating and sustaining openings there and for preventing restenosis thereof after implantation comprising a stent structure coated with a compound containing a high density, negatively charged domain of at least three vicinally oriented phosphorus-containing radicals.

The invention also relates to a coated stent with a compound comprising phosphorus-containing radicals with the following formula:

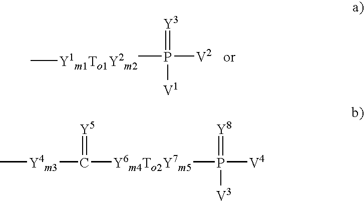

wherein

V¹ to V⁴ are Y⁸ₘ₆Tₒ₃U

Tₒ₁ to Tₒ₃ are (CH₂)ₙ, CHCH, or CH₂CHCHCH₂

o1 to o3 are 0 to 1

n is 0 to 4

U is R¹Y⁹ₘ₇, CY¹⁰Y¹¹R², SY¹²Y¹³Y¹⁴R³, PY¹⁵Y¹⁶Y¹⁷R⁴R⁵, Y¹⁸PY¹⁹Y²⁰Y²¹R⁶R⁷, CH₂NO₂, NHSO₂R⁸ or NHCY²²Y²³R⁹

m1 to m7 are 0 to 1

Y¹ to Y²³ are NR¹⁰, NOR¹¹, O or S

and where R¹ to R¹¹ are

i) hydrogen

ii) a straight or branched saturated or unsaturated alkyl residue containing 1-22 carbon atoms

iii) a saturated or unsaturated aromatic or non-aromatic homo- or heterocyclic residue containing 3-22 carbon atoms and 0-5 heteroatoms consisting of nitrogen, oxygen or sulfur

iv) a straight or branched saturated or unsaturated alkyl residue containing 1-22 carbon atoms substituted with a saturated or unsaturated aromatic or non-aromatic homo- or heterocyclic residue containing 3-22 carbon atoms and 0-5 heteroatoms consisting of nitrogen, oxygen or sulfur

v) an aromatic or non-aromatic homo- or heterocyclic residue containing 3-22 carbon atoms and 0-5 heteroatoms consisting of nitrogen, oxygen or sulfur substituted with a straight or branched saturated or unsaturated alkyl residue containing 1-22 carbon atoms.

in the said groups ii-v the residues and/or the substituents thereof being substituted with 0-6 of the following groups: hydroxy, alkoxy, aryloxy, acyloxy, carboxy, alkoxycarbonyl, alkoxycarbonyloxy, aryloxycarbonyl, aryloxycarbonyloxy, carbamoyl, fluoro, chloro, bromo, azido, cyano, oxo, oxa, amino, imino, alkylamino, arylamino, acylamino, arylazo, nitro, alkylthio or alkylsulfonyl.

The straight or branched saturated or unsaturated alkyl residue in groups i-v above can be exemplified by methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, heneicosyl, doeicosyl, isopropyl, isobutyl, isopentyl, isohexyl, isoheptyl, isooctyl, isononyl, isodecyl, isodoecosyl, 2-butyl, 2-pentyl, 2-hexyl, 2-heptyl, 2-octyl, 2-nonyl, 2-decyl, 2-doeicosyl, 2-methylbutyl, 2-methylpentyl, 2-methylhexyl, 2-methylheptyl, 2-methyloctyl, 2-methylnonyl, 2-methyldecyl, 2-methyleicosyl, 2-ethylbutyl, 2-ethylpentyl, 2-ethylhexyl, 2-ethylheptyl, 2-ethyloctyl, 2-ethylnonyl, 2-ethyldecyl, 2-ethyleicosyl, tertbutyl, ethenyl, propenyl, butenyl, pentenyl, hexenyl, heptenyl, octenyl, nonenyl, decenyl, undecenyl, dodecenyl, tridecenyl, tetradecenyl, pentadecenyl, hexadecenyl, heptadecenyl, octadecenyl, nonadecenyl, eicosenyl, heneicosenyl, doeicosenyl, butadienyl, pentadienyl, hexadienyl, heptadienyl, octadienyl, nonadienyl, decadienyl, doeicodienyl, ethynyl, propynyl, doeicosynyl.

The saturated or unsaturated aromatic or non-aromatic homo- or heterocyclic residue in groups i-v above can be exemplified by cyclopropyl, cyclobutyl, cyclopentyl, cyclohexyl, cycloheptyl, cyclooctyl, cyclononyl, cyclodecyl, cycloundecyl, cyclododecyl, cycloridecyl, cyclotetradecyl, cyclopentadecyl, cyclohexadecyl, cycloheptadecyl, cyclooctadecyl, cyclononadecyl, cycloeicosyl, cycloheneicosyl, cyclodoeicosyl, adamantyl, cyclopropenyl, cyclobutenyl, cyclopentenyl, cyclohexenyl, cycloheptenyl, cyclooctenyl, cyclononenyl, cyclodecenyl, phenyl, biphenyl, naphthyl, hydroxyphenyl, aminophenyl, mercaptophenyl, fluorophenyl, chlorophenyl, azidophenyl, cyanophenyl, carboxyphenyl, alkoxyphenyl, acyloxyphenyl, acylphenyl, oxiranyl, thiiranyl, aziridinyl, oxetanyl, thietanyl, azetidinyl, tetrahydrofuranyl, tetrahydrothiophenyl, pyrrolidinyl, tetrahydropyranyl, tetrahydrothiopyranyl, piperidinyl, quinuclidinyl, dioxanyl, dithianyl, trioxanyl, furyl, pyrrolyl, thienyl, pyridyl, quinolyl, benzofuryl, indolyl, benzothienyl, oxazolyl, imidazolyl, thiazolyl, pyridazinyl, pyrimidyl, pyrazinyl, purinyl or a carbohydrate.

Substituents may be selected from the group of: hydroxy, alkoxy, aryloxy, acyloxy, carboxy, alkoxycarbonyl, alkoxycarbonyloxy, aryloxycarbonyl, aryloxycarbonyloxy, carbamoyl, fluoro, chloro, bromo, azido, cyano, oxo, oxa, amino, imino, alkylamino, arylamino, acylamino, nitro, alkylthio, alkylsulfonyl.

Furthermore the invention relates to a coated stent wherein the phosphorus-containing radicals have the following formula:

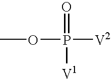

wherein V¹ and V² are OH, (CH₂)ₚOH, COOH, CONH₂, CONOH, (CH₂)ₚCOOH, (CH₂)ₚCONH₂, (CH₂)ₚCONOH, (CH₂)ₚSO₃H, (CH₂)ₚSO₃NH₂, (CH₂)ₚNO₂, (CH₂)ₚPO₃H₂, O(CH₂)ₚOH, O(CH₂)ₚCOOH, O(CH₂)ₚCONH₂, O(CH₂)ₚCONOH, (CH₂)ₚSO₃H, O(CH₂)ₚSO₃NH₂, O(CH₂)ₚNO₂, O(CH₂)ₚPO₃H₂, CF₂COOH and p is 1 to 4.

In this embodiment of the invention the phosphorus-containing radicals are phosphonates or phospates or derivatives thereof.

In one embodiment of the invention the backbone to the high density negatively charged region of vicinally oriented radicals is a cyclic moiety.

The cyclic moiety comprises a saturated or unsaturated aromatic or non-aromatic homo- or heterocyclic moiety. When the moiety is heterocyclic the heteroatoms are selected from the group of oxygen, nitrogen, sulfur or selenium.

Preferably the cyclic moiety comprises 4 to 24 atoms, most preferably 5 to 18 atoms. The cyclic moiety is for example selected from the group of cyclopentane, cyclohexane, cycloheptane, cyclooctane, inositol, monosacharide, disacharide, trisacharide, tetrasacharide, piperidin, tetrahydrothiopyran, 5-oxotetrahydrothiopyran, 5,5-dioxotetrahydrothiopyran, tetrahydroselenopyran, tetrahydrofuran, pyrrolidine, tetrahydrothiophene, 5-oxotetrahydrothiophene, 5,5-dioxotetrahydrothiophene, tetrahydroselenophene, benzene, cumene, mesitylene, naphtalene and phenantrene. When the cyclic moiety is an inositol it could be selected from the group of alloinositol, cisinositol, epiinositol, D/L-chiroinositol, scylloinositol, myoinositol, mucoinositol and neoinositol.

In one preferred embodiment of the intention the compounds are phosphates, phosphonates or phosphinates of cyclohexane such as 1,2,3-β-cyclohexane-1,2,3-trioltrisphosphate.

In other preferred embodiments of this type of the invention the compounds are phosphates, phosphonates or phosphinates of inositol. Preferably the number of phosphate, phosphonate or phosphinate radicals per inositol moiety is at least three. The remaining hydroxyl groups on the inositol moiety may be derivatized in the form of ethers or esters.

In one preferred embodiment the compound is myo-inositol-1,2,6-tris-phosphate, myo-inositol-1,2,3-trisphosphate or myo-inositol-hexa-kis phosphate.

In one most preferred embodiment the compounds are selected from the group of D-myo-inositol-1,2,6-trisphosphate, D-myo-inositol-1,2,6-tris(carboxymethylphosphate), D-myo-inositol-1,2,6-tris(carbomethylphosphonate), D-myo-inositol-1,2,6-tris (hydroxymethylphosphonate), D-3,4,5-tri-O-methyl-myo-inositol-1,2,6-trisphosphate, D-3,4,5-tri-O-hexanoyl-myo-inositol-1,2,6-trisphosphate, D-3,4,5-tri-O-butanoyl-myo-inositol-1,2,6-trisphosphate, D-3,4,5-tri-O-pentanoyl-myo-inositol-1,2,6-trisphosphate, D-3,4,5-tri-O-isobutanoyl-myo-inositol-1,2,6-trisphosphate, D-3,4,5-tri-O-propanoyl-myo-inositol-1,2,6-trisphosphate, D-3,4,5-tri-O-(6-hydroxy-4-oxa)hexanoyl-myo-inositol-1,2,6-trisphosphate, D-3,4,5-tri-O-3-(ethylsulphonyl)propanoyl-myo-inositol-1,2,6-trisphosphate, D-3,4,5-tri-O-3-hydroxypropanoyl-myo-inositol-1,2,6-trisphosphate, D-3,4,5-tri-O-(6-hydroxy)-hexanoyl-myo-inositol-1,2,6-trisphosphate, D-5-O-hexanoyl-myo-inositol-1,2,6-trisphosphate, D-3,4,5-tri-O-phenylcarbamoyl-myo-inositol-1,2,6-trisphosphate, D-3,4,5-tri-O-propanoyl-myo-inositol-1,2,6-tris(carboxymethylphosphate), D-3,4,5-tri-O-butanoyl-myo-inositol-1,2,6-tris (carboxymethylphosphate), D-3,4,5-tri-O-isobutanoyl-myo-inositol-1,2,6-tris(carboxymethyl-phosphate), D-3,4,5-tri-O-pentanoyl-myo-inositol-1,2,6-tris(carboxymethylphosphate), D-3,4,5-tri-O-hexanoyl-myo-inositol-1,2,6-tris(carboxymethylphosphate), D-3,4,5-tri-O-propanoyl-myo-inositol-1,2,6-tris(carboxymethylphosphonate), D-3,4,5-tri-O-butanoyl-myo-inositol-1,2,6-tris (carboxymethyl-phosphonate), D-3,4,5-tri-O-isobutanoyl-myo-inositol-1,2,6-tris(carboxymethylphosphonate), D-3,4,5-tri-O-pentanoyl-myo-inositol-1,2,6-tris(carboxymethylphospbonate), D-3,4,5-tri-O-hexanoyl-myo-inositol-1,2,6-tris (carboxymethylphosphonate), D-3,4,5-tri-O-propanoyl-myo-inositol-1,2,6-tris(hydroxymethyl-phosphonate), D-3,4,5-tri-O-butanoyl-myoinositol-1,2,6-tris(hydroxymethylphosphonate), D-3,4,5-tri-O-isobutanoyl-myo-inositol-1,2,6-tris(hydroxymethylphosphonate), D,-3,4,5-tri-O-pentanoyl-myo-inositol-1,2,6-tris(hydroxymethylphosphonate), D-3,4,5-tri-O-hexanoyl-myo-inositol-1,2,6-tris(hydroxymethyl-phosphonate).

When the cyclic moiety is a sacharide it could be selected from the group of D/L-ribose, D/L-arabinose, D/L-xylose, D/L-lyxose, D/L-allose, D/L-altrose, D/L-glucose, D/L-mannose, D/L-gulose, D/L-idose, D/L-galactose, D/L-talose, D/L-ribulose, D/L-xylulose, D/L-psicose, D/L-sorbose, D/L-tagatose and D/L-fructose or derivatives thereof. In preferred embodiments of this type of the invention the compounds are phosphates, phosphonates or phosphinates of sacharides. Preferably the number of phosphate, phosphonate or phosphinate radicals per sacharide unit is at least three. The remaining hydroxyl groups on the sacharide moiety may be derivatized in the form of ethers or esters. In many instances the ether form is desired as this type of radical prolongs the stability and half-life in vivo as the susceptibility to enzymatic degradation is reduced.

In one preferred embodiment of this type of the invention the compound is selected from the group of mannose-2,3,4-trisphosphate, galactose-2,3,4-trisphosphate, fructose-2,3,4-trisphosphate, altrose-2,3,4-trisphosphate and rhamnose-2,3,4-trisphosphate. In one most preferred embodiment the compound is selected from the group of R¹-6-O—R²-α-D-manno-pyranoside-2,3,4-trisphosphate, R¹-6-O—R²-α-D-galacto-pyranoside-2,3,4-trisphosphate, R¹-6-O—R²-α-D-altropyranoside-2,3,4-trisphosphate and R¹-6-O—R²-β-D-fructopyranoside-2,3,4-trisphosphate where R¹ and R² independently are as defined above and preferably are methyl, ethyl, propyl, butyl, pentyl or hexyl. Most preferred compounds in this type of the invention are methyl-6-O-butyl-α-D-mannopyranoside-2,3,4-trisphosphate, methyl-6-O-butyl-α-D-galactopyranoside-2,3,4-trisphosphate, methyl-6-O-butyl-α-D-glycopyranoside-2,3,4-trisphosphate, methyl-6-O-butyl-α-D-altropyranoside-2,3,4 trisphosphate, methyl-6-O-butyl-β-D-fructopyranoside-2,3,4-trisphosphate, 1,5-anhydro-D-arabinitol-2,3,4-trisphosphate, 1,5-anhydroxylitol-2,3,4-trisphosphate, 1,2-O-ethylene-β-D-fructopyranoside-2,3,4-trisphosphate, methyl-α-D-rhamno-pyranoside-2,3,4-trisphosphate, methyl-α-D-mannopyranoside-2,3,4-trisphosphate, methyl-6-O-butyl-α-D-mannopyranoside-2,3,4-tris-(carboxymethylphosphate), methyl-6-O-butyl-α-D mannopyranoside-2,3,4-tris(carboxymethylphosphonate), methyl-6-O-butyl-α-D-mannopyranoside-2,3,4-tris(hydroxymethyl-phosphonate), methyl-6-O-butyl-α-D-galactopyranoside-2,3,4-tris(carboxymethylphosphate), methyl-6-O-butyl-α-D-galacto-pyranoside-2,3,4-tris(carboxymethylphosphonate), methyl-6-O-butyl-α-D-galactopyranoside-2,3,4-tris(hydroxymethyl-phosphonate), methyl-6-O-butyl-α-D-glucopyranoside-2,3,4-tris (carboxymethylphosphate), methyl-6-O-butyl-α-D-glucopyranoside-2,3,4-tris(carboxymethylphosphonate), methyl-6-O-butyl-α-D-glucopyranoside-2,3,4-tris (hydroxymethyl-phosphonate), methyl-6-O-butyl-α-D-altropyranoside-2,3,4-tris-(carboxymethylphosphate), methyl-6-O-butyl-α-D-altropyranoside-2,3,4-tris-(carboxymethylphosphonate), methyl-6-O-butyl-α-D-altropyranoside-2,3,4-tris-(hydroxymethylphosphonate), methyl-6-O-butyl-α-D-fructopyranoside-2,3,4-tris-(carboxymethylphosphate), methyl-6-O-butyl-α-D-fructopyranoside-2,3,4-tris-(carboxymethylphosphonate), methyl-6-O-butyl-α-D-fructopyranoside-2,3,4-tris-(hydroxymethylphosphonate).

In other preferred embodiments of the invention the compounds are phosphates, phosphonates or phosphinates of heterocyclic moieties such as 1,5-dideoxy-1,5-iminoarabinitol-2,3,4-trisphosphate, 1,5-dideoxy-1,5-iminoarabinitol-2,3,4-tris-(carboxymethylphosphate), 1,5-dideoxy-1,5-imino-arabinitol-2,3,4-tris(carboxymethylphosphonate), 1,5-dideoxy-1,5-iminoarabinitol-2,3,4-tris(hydroxymethylphosphonate), 1,5-dideoxy-1,5-imino-N-(2-phenylethyl)arabinitol-2,3,4-trisphosphate, 1,5-dideoxy-1,5-imino-N-(2-phenylethyl)-arabinitol-2,3,4-tris(carboxymethylphosphate), 1,5-dideoxy-1,5-imino-N-(2-phenylethyl)arabinitol-2,3,4-tris-(carboxymethylphosphonate), 1,5-dideoxy-1,5-imino-N-(2-phenyl-ethyl) arabinitol-2,3,4-tris (hydroxymethylphosphonate).

Within the process of restenosis, a number of regulatory substances are released. One category of substances are growth factors and the activity of these substances are considered to promote cell proliferation, neointimal formation and hyperplasia. Ligand-induced dimerisation is a key event in transmembrane signalling by receptors with tyrosinase kinase activity. Unlike other growth factors such as platelet derived growth factor (PDGF) which are dimeric, fibroblast growth factors (FGF) are monomeric and are unable by themselves to induce activation of FGF receptors. Accordingly, FGF function in concert with for example heparin which induce dimerization and subsequent activation which leads to for example induction of transcriptional factors like early growth response factor 1 (Egr-1). It has been hypothesised that Egr 1 may play a key regulatory role by linking injurious stimuli to the induction of genes directing the expression of effector molecules in endothelial cells, smooth muscle cells, fibroblasts and leukocytes.

Such a complex between fibroblast growth factor 2 (FGF2) and its naturally occurring receptor 1 (FGFR1) has been structurally determined and it can be observed that a positively charged canyon formed by a cluster of basic amino acid residues represent the heparin-binding site. One type of activity of the described compounds according to the invention is to replace or take the place of heparin in this domain and thereby act as a blocking agent of fibroblast growth receptors. The consequence of this activity is to counteract the dimerization and subsequent activation, which leads to a down regulation of the detrimental effects of released substances and thereby a halt in the process of restenosis. Furthermore, another activity of the compounds according to the present invention is to affect the vascular remodelling which is an important factor in the process of restenosis. Remodelling is characterised by thickening and enlargement of existing cells and tissue. The geometry of vessels such as arteries can normally change in response to alterations in the local environment but the process of remodelling counteracts this compensatory response. When administering the compounds according to the invention, one effect is that the process of remodelling is counteracted evidenced by a diminished transformation of fibroblasts into myofibroblasts, positive effects on collagen synthesis and a reduction of the deposition of extracellular matrix. Furthermore, an improved elasticity of the tissue and vessels are observed which shows a positive effect on the cytoskeletal structure of the vessels.

According to the invention the compounds are most often present in a salt form or in a form where only a few of the negative charges are protonated. The salt can contain one or more cations in different combinations. Examples of cations are sodium and potassium ions.

Formulations could comprise the active compound per se but also in a mixture with non-toxic pharmaceutically acceptable carriers, excipients and diluents. These may be, for example, buffers, antioxidants, glucose, sucrose, dextrins, albumin and so forth.

Naturally, the therapeutic dosage range for the compounds of the present invention will vary with the particular condition or disease symptom being treated with the surgical stent.

According to the present invention the use of a restenosis resistent stent implantation to a human patient comprising the steps of selecting a coated stent for implantation in human vessels, orifices and conduits for creating and sustaining openings there and for preventing, alleviating or combatting restenosis thereof after implantation, comprising a stent structure coated with a compound containing a high density, negatively charged domain of at least three vicinally oriented phosphorus-containing radicals is also described.

The invention also relates to the use of a restenosis resistent stent which is coated with a compound containing phosphorus-containing radicals with the following formula as described above:

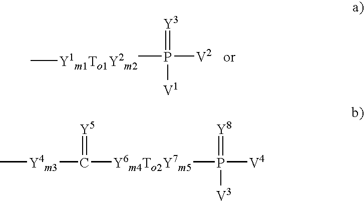

Furthermore, the invention also relates to the use of a restenosis resistent stent which is coated with a compound containing phosphorus-containing radicals with the following formula as described above:

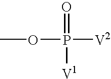

A variety of embodiments are considered according to the invention wherein the lumen of a body passageway is expanded to eliminate a biliary, esophageal, tracheal, broncheal, urethral or vascular obstruction and whereby the use of a coated stent with a compound containing phosphorus-containing radicals as defined above has a benefical effect against restenonsis or other conditions where counteracting cell proliferation, neointimal formation and hyperplasia is considered to be essential.

The invention will be further explained with the following embodiment examples however without limiting it thereto.

Example 1 and 2 describe the counteractive effects of D-myo-inositol-1,2,6-trisphosphate (IP₃) and D-3,4,5-tri-O-(phenylcarbamoyl)myo-inositol-1,2,6-trisphosphate (PP 11-201) on the proliferation of smooth muscle cells. Example 3 and 4 show the inhibitory effects of D-myo-inositol-1,2,6-trisphosphate (IP₃) and D-3,4,5-tri-O-(phenylcarbamoyl)myo-inositol-1,2,6-trisphosphate (PP 11-201) on neointima formation. Example 5 discloses the interaction between the complex of Fibroblast Growth Factor 2-Fibroblast Growth Factor Receptor 1 (FGF2-FGFR1) and D-myo-inositol-1,2,6-trisphosphate. Example 6 shows the coating of a metal stent with a polymeric matrix containing D-3,4,5-tri-O-hexanoyl-myo-inositol-1,2,6-trisphosphate (PP 10-202). Example 7 demonstrates the safety profile of a series of compounds according to the invention.

### EXAMPLE 1

Vascular smooth muscle cell proliferation contributes to restenosis after coronary angioplasty. Human pulmonary artery smooth muscle cells (HPASMCs) were maintained in Medium 231 supplemented with Smooth muscle Growth Supplement. The cells, medium and supplement were obtained from Caascade Biologics Inc., Portland, Oreg., USA.

The cells were grown as monolayer culture in 100-mm diameter plastic tissue culture dishes (Nunc, Roskilde, Denmark) with 10 ml medium in 37.degree. C. in a humidified atmosphere of 95% air and 5% CO₂ and subcultured at 4- to 6-day intervals. For measurement of cell growth, cells were seeded at density 5.0*10³/well in 96 well dishes in DMEM+0.2% FBS for 3 days. Culture medium was then removed and cells were seeded in DMEM+0.2% FBS with 1 nM of basic fibroblast growth factor (FGF2). The addition of FGF2 induces cell proliferation of the HPASMCs. The FGF2 used was a recombinant human substance from Boehringer Mannheim Biochemica, Espoo, Finland. Various concentrations of D-myo-inositol-1,2,6-trisphosphate (IP₃) were added and the number of parallel wells in each treatment was four. The proliferation of cells was analysed after 24 hours of incubation by labelling each well with 0.2 μCi of ³H-thymidine for two hours. After that the cells were trypsinated and harvested on a filter. A Melt-on scintillator (Meltilex™ A; Wallac, Turku, Finland) was allowed to melt on the filter. The radioactivity of the sheets was counted on a liquid scintillator counter (1450 Microbeta Wallac, Turku, Finland).

Addition of FGF2 significantly increased the incorporation of ³H-thymidine in DNA in HPASMCs, which is the measurement of the proliferation of the cells. Stimulation of proliferation was inhibited by the addition of IP₃ with the following result:

| Substance | concentration (μM) | inhibition (%) |
|---|---|---|
| IP₃ | 1 | 61 |
| | 10 | 44 |
| | 100 | 68 |

The experiment shows that there is a strong effect of IP₃ to inhibit stimulated proliferation of smooth muscle cells.

### EXAMPLE 2

In a procedure similar to the one described in Example 1, D-3,4,5-tri-O-(phenylcarbamoyl) myo-inositol-1,2,6-trisphosphate (PP 11-201) was added with the result shown below:

| Substance | concentration (μM) | inhibition (%) |
|---|---|---|
| PP 11-201 | 0.1 | 49 |
| | 1.0 | 61 |
| | 10 | 70 |

The experiment shows that there is a strong effect of PP 11-201 to inhibit stimulated proliferation of smooth muscle cells.

### EXAMPLE 3

The effects on neointima formation was assessed after inducing vessel injury in rats with a balloon catheter. Neointima formation is an important part in the process of restenosis. Adult mate Wistar rats (250-260 g) were housed under standard conditions and were fed with commercial rat chow and water ad libitum. During the experiment D-myo-inositol-1,2,6-trisphosphate (IP₃) was administered via subcutaneously operated osmotic minipumps with a dose level of 1 mg/kg/hr. A control group received saline solution by osmotic minipumps. The animals were anaesthetised with an intraperitoneal injection of ketamine, 100 mg/kg, (MTC Pharmaceuticals, Cambridge, Ontario, Canada) and xylazine, 10 mg/kg (Bayer Inc., Etobicoke, Ontario, Canada). Using a dissecting microscope, the left carotid artery was exposed on the ventral side of the neck via a midline incision. The bifurcation of the carotid artery was located and two ligatures were placed around the external carotid artery, which was then tied off with the distal ligature. After temporarily occluding the internal carotid artery with a vascular clamp, a small incision was made between the two ligatures placed around the external carotid artery to introduce the endothelial denudation device, an inflated French Fogarty embolectomy catheter (Baxter Healthcare, Buckinghamshire, UK). Each animal was de-endothelialised by three passages of the catheter and the carotid artery was tied off proximal to the incision hole. The clamp was removed and the pulse of the carotid artery was rechecked. The skin incision was closed with a single suture. 14 days post-operatively, the animals were sedated. An abdominal incision was made to access the abdominal aorta for insertion of a cannula connected to a perfusion apparatus. The animals where killed by an overdose of anaestetic and then perfused with heparinised phosphate buffered saline (pH 7.4) at a rate of 100 ml/min per kg body weight and a pressure of 120 mm Hg. After replacement of saline, 4% paraformaldehyde in isotonic saline was introduced at the same flow rate. After fixation in situ, the carotids were dissected free. Three mid-carotid segments, approximately 10 mm long were rinsed and placed in 4% parafomaldehyde for another 16 hours before embedding and freezing in Tissue-Tek OCT media (Miles Inc., Elkhart, Ind., USA). Frozen sections were stained with Movat's pentachrome and immunohistochemistry was performed using antibodies in order to examine the process of restenosis by measurement of the neointima formation. It was observed that the administration of IP₃ reduced the formation as can be seen in the following table:

| Substance | Neointima formation (mm²) | Reduction (%) |
|---|---|---|
| Control | 1.12 | |
| IP₃ | 0.48 | 57 |

The experiment shows that the administration of IP₃ reduced neointima formation with 57%, which describes a beneficial effect against restenosis. By microscopic investigation it could be observed that the elasticity of the vessel walls were improved in the animals receiving IP₃ compared to the vessel walls of the control group which shows a positive effect on remodelling.

### EXAMPLE 4

In a procedure similar to the one described in Example 3, D-3,4,5-tri-O-(phenylcarbamoyl)myo-inositol-1,2,6-trisphosphate (PP 11-201) was added with the result shown below:

| Substance | Neointima formation (mm²) | Reduction (%) |
|---|---|---|
| Control | 1.12 | |
| PP 11-201 | 0.43 | 62 |

The experiment shows that the administration of PP 11-201 reduced neointima formation with 62%, which describes a beneficial effect against restenosis. By microscopic investigation it could be observed that the elasticity of the vessel walls were improved in the animals receiving PP 11-201 compared to the vessel walls of the control group which shows a positive effect on remodelling.

### EXAMPLE 5

The interaction between the complex of Fibroblast Growth Factor 2-Fibroblast Growth Factor Receptor 1 (FGF2-FGFR1) and a model compound, D-myo-inositol-1,2,6-trisphosphate, was studied with the Insight modelling package on Silicon Graphics platform using a manual 3-dimensional docking procedure. The protein X-ray structure of FGF2-FGFR1 was according to Plotnikov et al, Cell 98, 641 (1999), Protein Data Bank entry 1CVS.

A long cleft of the protein complex (30. ANG.) exposes two binding sites to the model compound with the following characteristics:

### Site 1.

Near the central part of the cleft there is a concentration of positively charged residues: Lys A26, Lys A135, Lys C163, Lys C166, Lys C172, Lys C175, Lys C207, Arg A120 and His C166. The following distances between the oxygen in the P—O radical in the model compound and the nitrogen atoms in the positively charged residues were characterized:

| Residue | Distance (Å) |
|---|---|
| Lys C163 | 2.4 |
| Lys A135 | 2.7 |
| Lys C172 | 2.7 |
| His C166 | 2.3 |

### Site 2.

Near the end of the cleft region there is a concentration of another set of positively charged residues: Lys B26, Lys B135, Lys B120, Lys D172, Lys C175, Lys D163, Lys C207, Arg B120 and His D166. The following distances between the oxygen in the P—O radical in the model compound and the nitrogen atoms in the positively charged residues were characterized:

| Residue | Distance (Å) |
|---|---|
| Lys B135 | 2.3 |
| Lys D163 | 3.6 |
| Lys C175 | 3.2 |
| His D166 | 3.2 |

The modelling experiment shows a strong binding between the model compound, D-myo-inositol-1,2,6-trisphosphate, and two distinct sites of the FGF2-FGFR1 receptor complex.

### EXAMPLE 6

A solution of polyethylene vinyl acetate (EVA, 2% w/w in dichloromethane and 0.2% of PP 10-202 was prepared. The stent is dipped once into the EVA-PP 10-202 solution and is allowed to dry in order to yield a smooth uniform coating. In order to obtain a thicker coating the dipping process can be repeated or the concentration of the EVA-PP 10-202 solution can be varied. Typically, the polymer concentration can be varied from 0.1% to 10% and the content of PP 10-202 can be varied between 1 to about 20% of the polymer weight. The polymer carrier can be any pharmaceutically acceptable biopolymer that is non-degradable and insoluble in biological mediums, has good stability in a biological environment, has good adherence to the selected stent, is flexible and that can be applied as coating to the surface of a stent either from an organic solvent or by a melting process. The hydrophilicity or hydrophobicity of the polymer carrier will determine the release rate of PP 10-202 from the stent surface. Hydrophilic polymers such as copolymers of hydroxyethyl methacrylate-methyl methacrylate and segmented polyurethane may be used. Hydrophobic coatings such as copolymers of ethylene vinyl acetate, silicone colloidal solutions and polyurethanes may be used.

The preferred polymers would be those that are rated as medical grade and that are having good compatibility in contact with blood.

### EXAMPLE 7

A method described by S. Irwin (Psychopharmacologica, 1968, 13, 222) was used to detect general physiological, behavioural and toxic effects of a series of compounds. The tested compounds were administered intra veneously to mice (3 animals per compound and dose) and the behavioural modification and neurotoxicity symptoms such as mortality, sedation, excitation, aggressiveness, straub, writhes, convulsions, tremor, exophtalamos, salivation, lacrimation, piloerection, defecation, fear, traction, reactivity to touch, loss of righting reflex, sleep, motor incoordination, muscle tone, stereotypies, catalepsy, grasping, ptosis, difficulty in respiration, corneal reflex, analgesia and gait were recorded compared to a control group, Furthermore, rectal temperature and pupil diameter were recorded.

The results are shown in the following table:

| | Dose where effects |
|---|---|
| Compound | are observed (mg/kg) |
| D-myo-inositol-1,2,6-trisphosphate | >512 |
| D-3,4,5-tri-O-pentanoyl- | 128 |
| myo-inositol-1,2,6-trisphosphate | |
| D-3,4,5-tri-O-propanoyl- | 256 |
| myo-inositol-1,2,6-trisphosphate | |
| D-myo-inositol-1,2,6- | >512 |
| tris(carboxymetylphosphonate) | |
| methyl-6-O-butyl-α-D-glycopyranoside- | 256 |
| 2,3,4-trisphosphate | |
| methyl-6-O-butyl-α-D-mannopyranoside- | >512 |
| 2,3,4-trisphosphate | |
| 1,2-O-ethylene-β-D-fructopyranoside- | >512 |
| 2,3,4-trisphosphate | |
| 1,5-dideoxy-1,5-imino-N- | >512 |
| (2-phenylethyl)arabinitol-2,3,4-trisphosphate | |

From the data it can be concluded that the compounds have a very good safety profile which is beneficial for the use according to invention.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of the equivalence of the claims are therefore intended to be embraced therein.

What is claimed:

1. A method of implanting a stent whereby the risk of restenosis is reduced, the method comprising:
implanting into a patient susceptible to post-implant restenosis a stent coated with a therapeutically effective amount of a compound for reducing the onset or severity of restenosis, the compound containing a high density, negatively charged domain of at least three vicinally oriented phosphorus-containing radicals.

2. The method of claim 1, wherein the phosphorus-containing radicals have the following formula: 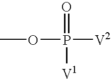
wherein V¹ and V² are the same or different in the above formula and are each independently selected from the group consisting of OH, (CH₂)ₚOH, COOH, CONH₂, CONOH, (CH₂)ₚCOOH, (CH₂)ₚCONH₂, (CH₂)ₚCONOH, (CH₂)ₚSO₃H, (CH₂)ₚSO₃ NH₂, (CH₂)ₚNO₂, (CH₂)ₚPO₃H₂, O(CH₂)ₚOH, O(CH₂)ₚCOOH, O(CH₂)ₚCONH₂, O(CH₂)ₚCONOH, (CH₂)ₚSO₃H, O(CH₂)ₚSO₃NH₂, O(CH₂)ₚNO₂, O(CH₂)ₚPO₃H₂, and CF₂COOH, wherein p is a value from 1 to 4.

3. The method of claim 2, wherein the phosphorus-containing radicals are phosphate groups.

4. The method of claim 1, wherein a backbone to the high density, negatively charged domain of the at least three vicinally oriented phosphorus-containing radicals is a cyclic moiety.

5. The method of claim 4, wherein the backbone is a saturated or unsaturated aromatic or non-aromatic homo- or heterocyclic moiety where the heteroatom is nitrogen, oxygen, sulfur or selenium.

6. The method of claim 5, wherein the saturated or unsaturated aromatic or non-aromatic homo- or heterocyclic moiety comprises 4 to 24 atoms.

7. The method of claim 6, wherein the saturated or unsaturated aromatic or non-aromatic homo- or heterocyclic moiety is selected from the group consisting of cyclopentane, cyclohexane, cycloheptane, inositol, monosaccharide, disaccharide, trisaccharide, tetrasaccharide, piperidine, tetrahydrothiopyran, 5-oxotetrahydrothiopyran, 5,5-dioxotetrahydrothiopyran, tetrahydroselenophyran, tetrahydrofuran, pyrrolidine, tetrahydrothiophene, 5-oxotetrahydrothiophene, 5,5-dioxotetrahydrothiophene, tetrahydroselenophene, benzene, cumene, mesitylene, naphthalene, and phenanthrene.

8. The method of claim 7, wherein the saturated or unsaturated aromatic or non-aromatic homo- or heterocyclic moiety is an inositol selected from the group consisting of alloinositol, cisinositol, epiinositol, D/L-chiroinositol, scylloinositol, myoinositol, mucoinositol and neoinositol.

9. The method of claim 7, wherein the saturated or unsaturated aromatic or non-aromatic homo- or heterocyclic moiety is a monosaccharide selected from the group consisting of D/L-ribose, D/L-arabinose, D/L-xylose, D/L-lyxose, D/L-allose, D/L-altrose, D/L-glucose, D/L-mannose, D/L-glucose, D/L-idose, D/L-galactose, D/L-talose, D/L-ribulose, D/L-xylulose, D/L-psicose, D/L-sorbose, D/L-tagatose and D/L-fructose.

10. The method of claim 2, wherein one of the phosphorus-containing radicals is axial and two of the phosphorus-containing radicals are equatorial.

11. The method of claim 1, wherein the compound is selected from the group consisting of myo-inositol-1,2,6-trisphosphate, myo-inositol-hexa-kis-phosphate, mannose-2,3,4-trisphosphate, rhamnose-2,3,4-trisphosphate, galactose-2,3,4-trisphosphate, methyl-6-O-butyl-α-D-mannopyranoside-2,3,4-trisphosphate, 1,5-anhydro-D-arabinitol-2,3,4-trisphosphate, fructose-2,3,4-trisphosphate, 1,2-O-ethylene-β-D-fructopyranoside-2,3,4-trisphosphate, cyclohexane-1,2,3-triol trisphosphate, 1,5-dideoxy-1,5-iminoarabinitol-2,3,4-trisphosphate, altrose-2,3,4-trisphosphate, and methyl-6-O-butyl-α-D-altropyranoside-2,3,4-trisphosphate.

12. The method of claim 1, wherein the patient is at a reduced risk of restenosis compared to a patient with an implanted stent that is not coated with the compound.

13. The method of claim 1, wherein the stent is implanted into a body passage-way of the patient selected from the group consisting of vessel, orifice, and conduit.

14. The method of claim 1, wherein the compound coated on the stent inhibits the proliferation of smooth muscle cells after the implanting.

15. The method of claim 1, wherein the compound coated on the stent inhibits neointima formation after the implanting.

16. The method of claim 1, wherein the stent is coated with a formulation comprising the compound in a mixture with a non-toxic pharmaceutically acceptable carrier, excipient, or diluent.

17. The method of claim 16, wherein the pharmaceutically acceptable carrier, excipient, or diluent is selected from the group consisting of buffers, antioxidants, glucose, sucrose, dextrins, and albumin.

18. The method of claim 1, wherein the implanting results in expanding of a body passage-way to eliminate an obstruction selected from the group consisting of biliary, esophageal, tracheal, bronchial, urethral, and vascular obstruction.

19. The method of claim 1, wherein the stent is implanted in the patient for a time period between 7 and 30 days.

20. The method of claim 1, wherein the compound prevents the formation of restenotic lesions on the border between the stent and an untreated portion of a vessel or tubular wall after implantation.

21. The method of claim 1, wherein the stent was prepared by dipping the stent into a solution comprising the compound and allowing the stent to dry.
